(19) United States
(12) Patent Application Publication
(10) Pub. No.: US 2026/0314548 A1
(43) Pub. Date: Oct. 15, 2026

(54) SOIL MOISTURE APPARATUS FOR PLANTS

(71) Applicant: Rudolf Brandowski, Paderborn (DE)

(72) Inventors: Rudolf Brandowski, Paderborn (DE); Peter Poradisch, Paderborn (DE)

(21) Appl. No.: 18/998,851

(22) PCT Filed: Sep. 9, 2022
(86) PCT No.: PCT/EP2022/075064

§ 371 Date: Jan. 27, 2025

(30) Foreign Application Priority Data
Jul. 28, 2022 (DE) 10 2022 118 908.2

(51) Int. Cl. A01G 25/16; A01G 9/02; G01N 27/04; G01N 33/24

(52) U.S. Cl. CPC A01G 25/167; A01G 9/02; G01N 27/048; G01N 33/246

(57) ABSTRACT

A soil moisture apparatus for plants, having a housing for accommodating the plants and having a moisture measuring device containing a first capacitor electrode and a second capacitor electrode and an evaluation device, wherein the housing is of multi-part design and contains an inner pot for accommodating the plant soil and a cover pot that surrounds the inner pot on a base wall thereof and/or on a side wall thereof that rises from the base wall, or an inner pot for accommodating the plant soil and an intermediate pot that surrounds the inner pot on a base wall thereof and/or on a side wall thereof, and a cover pot that surrounds the intermediate pot on a base wall thereof and on a side wall thereof, and the first capacitor electrode and the second capacitor electrode are arranged on an inside of a base wall and/or of a side wall of the cover pot and/or of the intermediate pot, which inside faces the inner pot.

The invention relates to a soil moisture apparatus for plants according to the preamble of Claim 1.

EP 3 145 292 B1 discloses a moisture measuring device for plants that comprises an evaluation device and two capacitive electrodes. The capacitive electrodes are inserted into the potting compost in the plant pot.

DE 10 2020 118 565 A1 discloses a moisture measuring device for plants that has spacedly arranged capacitive electrodes. These are inserted into the potting compost in a plant pot. The drawback of the known measurement device is that it gets in the way or its position is undesirably modified when the plant pot is watered.

DE 20 2016 005 957 U1 discloses a soil moisture measuring device for plants with a housing for accommodating potting compost and with a moisture measuring device containing a first capacitive electrode and a second capacitive electrode, and an evaluation device. The capacitive electrodes and the evaluation device are arranged in a bottom region of the housing. The drawback of this is that a relatively large volume is required.

The object of the present invention is accordingly to further develop a soil moisture apparatus in such a way that convenience and ease of use are improved.

The invention achieves this object with the features of Claim 1.

The particular advantage of the invention is that the multi-part construction of a housing for accommodating the plant provides a physical separation between the actual plant or potting compost on the one hand and the moisture measuring device on the other. The plant is planted in a potting compost that is surrounded by an inner pot. The two capacitive electrodes are arranged on the outer side of a cachepot surrounding the inner pot or on the outer side of an intermediate pot. The standard commercial inner pot containing the plant and the potting compost can be straightforwardly inserted into the cachepot or into the intermediate pot without there being any need for additional fastening of parts of the moisture measuring device.

In a preferred embodiment of the invention, a first capacitive electrode and/or a second capacitive electrode is/are large in area, specifically covering a circumferential angle of at least 40° and preferably ranging from 160° to 175°. The circumferential angle extends about an axis of symmetry of the cachepot or inner pot. The electrodes preferably each extend from a bottom wall to an upper opening edge of the cachepot or intermediate pot. The first capacitive electrode and the second capacitive electrode preferably extend over an identically sized area. This advantageously makes it possible to achieve a good measurement result without getting in the user's way during treatment (watering) of the plant.

According to a further development of the invention, the first capacitive electrode and the second capacitive electrode each take the form of an electrically conductive foil/film that is bonded to an outer side of the cachepot or intermediate pot. Advantageously, the electrodes together with their supply leads are consequently arranged compactly and protected from contact/damage by the wall of the cachepot or intermediate pot.

According to a further development of the invention, the cachepot is of two-part construction with a first part and a second part surrounded by the first part. The second part has the capacitive electrodes and is preferably arranged in an upper portion of the first part. A chamber for accommodating the evaluation device is provided in a lower portion of the first part. The first part thus surrounds the second part, the two parts preferably ending flush at an upper opening edge. The first part is constructed such that the evaluation device is fastened to the first part at an upper edge thereof. A second part could be constructed such that the evaluation device is located directly at the upper edge.

According to a further development of the invention, the second part of the cachepot has opposing grooves extending in a vertical direction on the inner side. The grooves are free of electrodes or bound the edges of the capacitive electrodes. The grooves thus advantageously define the arrangement of the electrodes on the outer side of the first part.

According to a further development of the invention, electrical cables that are connectable to terminals of the evaluation device protrude from an intermediate pot, which has the first capacitive electrode and the second capacitive electrode on the inner side or outer side or within it. The intermediate pot serves exclusively as a carrier for the first capacitive electrode, for the second capacitive electrode and for the electrode connection cables. The cachepot serves to accommodate the evaluation device and to accommodate the intermediate pot, wherein an inner side of the cachepot rests flat against an outer side of the intermediate pot. The intermediate pot serves to accommodate the inner pot which holds the plant.

According to a further development of the invention, the evaluation device has a solar panel and/or a supercapacitor, by way of which the evaluation device is supplied with electric power. Energy can advantageously be saved as a consequence.

Exemplary embodiments of the invention are explained in greater detail below with reference to the drawings.

In the drawings:

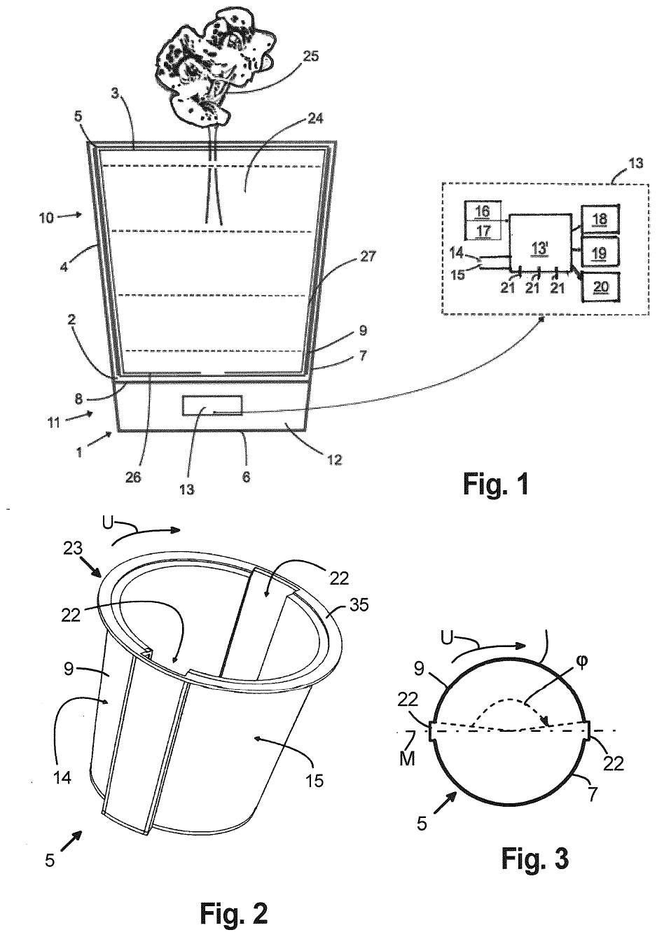

FIG. 1 is a schematic representation of a soil moisture apparatus according to a first embodiment, wherein a cachepot of the soil moisture apparatus is of two-part construction,

FIG. 2 is a perspective representation of a second, inner part of the cachepot according to FIG. 1,

FIG. 3 is a plan view of the second part according to FIG. 2,

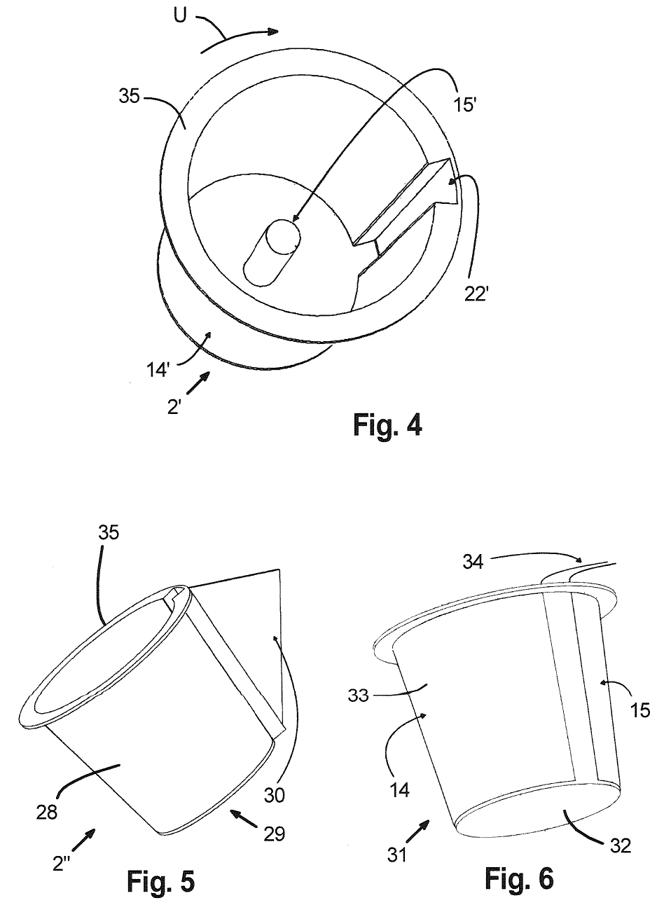

FIG. 4 is a representation of a one-piece cachepot according to a further embodiment of the invention,

FIG. 5 is a side view of a one-piece cachepot according to a further embodiment of the invention and

FIG. 6 is a perspective representation of an intermediate pot of the invention.

A soil moisture apparatus for plants has a housing for accommodating the plant 25 and potting compost 24, and a moisture measuring device 1.

According to the invention, the housing is of multi-part construction, it having a cachepot 2 on the one hand and an inner pot 3 on the other. The inner pot 3 serves to accommodate the potting compost 24 and the plant 25.

The cachepot 2 according to the embodiment of FIGS. 1 to 3 is of two-part embodiment and has a first part 4 and a second part 5 surrounded by the first part 4. The inner pot 3 may be a standard commercial plant pot made of stoneware or plastics or the like of any desired design. The first part 4 has a bottom wall 6 and a side wall 7 extending upright therefrom. The side wall 7 may be cylindrical or frustoconical in shape.

The second part 5 is, like the first part 4, pot-shaped and provided with a bottom wall 8 and an upright side wall 9. The second part 5 is constructed to be insertable with clearance into the first part 4.

The second part 5 is arranged in an upper portion 10 of the first part 4, said upper portion 10 being arranged on a side remote from the bottom wall 6 of the first part 4. Arranged in a lower portion 11, proximal to the bottom wall 6, of the first part 4 is a chamber 12, in which is arranged an evaluation device 13 of the moisture measuring device 1. The evaluation device 13 comprises electronics 13′ with a microprocessor and a program for programming a determination of watering needs and/or for automatic watering of the inner pot 3. The evaluation device 13 may, for example, generate a signal when the moisture content of the monitored potting compost 24 falls to a critical threshold value. In order to measure the moistness of the potting compost 24, the evaluation device 13 has a first capacitive electrode 14 and a second capacitive electrode 15 which are integrated into the second part 5 of the cachepot 2. The two electrodes 14, 15 are connected to the evaluation device 13 via supply leads (not shown).

A solar panel 16 and/or a supercapacitor 17 is/are provided to power the evaluation device 13. The solar panel 16 is arranged outside the cachepot, for example next to it, and can, for example, charge the supercapacitor 17. Alternatively, the solar panel 16 is located within the cachepot 2 or the first part 4. If the first part 4 is of cuboidal and/or polygonal construction, a rigid solar panel 16 is arranged flat against an outer side of the first part 4. If the first part 4 is of arcuate construction, a flexible solar panel 16 is arranged flat against an outer side of the first part 4.

The evaluation device 13 may, for example, have an evaluation program such that the moisture content of the potting compost 24 is measured once daily. This moisture value is stored in a memory of the evaluation device 13 until the next measurement. The evaluation device 13 is connected to an optical signaling device 18 which may, for example, have an LED light source for signaling a dry condition of the potting compost 24. If, for example, the moisture content falls below the threshold value, the color of the light source changes, for example, from green to red. Alternatively, the light source can also be monochromatic. The dry condition of the potting compost 24 can then, for example, be indicated by an illumination frequency of the light source. This signals to the user that liquid must be supplied to the potting compost 24.

The evaluation device 13 can furthermore be connected to a radio module 19 such that the dry condition can be transmitted, for example via WLAN, to a mobile data device.

Water can be supplied and apportioned to the potting compost 24 by the evaluation device 13 being connected to a switching element 20, for example a solenoid valve or a pump for delivering water from an external water reservoir to the potting compost 24 in the inner pot 3.

The evaluation device 13 can furthermore have separating resistors and/or inductors 21, such that signals can be provided for testing purposes and for simultaneously minimizing any interfering effects caused by the measuring leads used.

As is apparent from FIG. 2, the first capacitive electrode 14 and the second capacitive electrode 15 are arranged on an outer side of the second part 5 of the cachepot 2. The first capacitive electrode 14 and the second capacitive electrode 15 are large in area and preferably have the same dimensions. The first capacitive electrode 14 and the second capacitive electrode 15 are arranged opposite one another on the outer side of the side wall 9 of the second part 5. They are in each case isolated from one another merely by an upright groove 22, wherein the groove 22 extends on the inner side of the side wall 9 from the bottom wall 8 to a free opening edge 23 of the inner part 5.

The first capacitive electrode 14 and the second capacitive electrode 15 each extend over a circumferential angle □ of at least 40° and preferably ranging from 160° to 174°. In the present exemplary embodiment, the first capacitive electrode 14 and the second capacitive electrode 15 each extend over a circumferential angle □ of 175°. The circumferential angle □ extends in the circumferential direction U about an axis A of the second part 5. If the second part 5 is cylindrical in shape, the first capacitive electrode 14 and the second capacitive electrode 15 adopt the shape of parts of a cylinder. The first capacitive electrode 14 and the second capacitive electrode 15 extend symmetrically to one another with respect to a center plane M of the second part 5.

The first capacitive electrode 14 and the second capacitive electrode 15 each take the form of an electrically conductive foil/film, for example aluminum foil. They are bonded to the outer side of the second part 5, for example by adhesive (self-adhesive foil/film). The capacitive electrodes 14, 15 may alternatively be formed by applying electrically conductive substances, for example coatings.

The inner pot 3, which contains the potting compost 24 and the plant 25, preferably consists of a plastics material with a bottom wall 26 and a side wall 27 extending upright therefrom. The bottom wall 26 conventionally has perforations such that moisture or water can also be supplied to the potting compost 24 from below, for example in the case of orchids.

The second part 5 of the cachepot 2 is constructed such that the inner pot 3 can be inserted with clearance therein.

A further embodiment of the invention according to FIG. 4 provides a one-piece cachepot 2′ in which just one first capacitive electrode 14′ is arranged on the outer side of the cachepot 2′. A second capacitive electrode 15′ protrudes from a bottom wall 8′ of the cachepot 2′, wherein it is preferably located in a sleeve connected in one piece to the cachepot 2′. The first capacitive electrode 14′ extends over a circumferential angle □ ranging between 272° and 355°. The first capacitive electrode 14′ is interrupted by just one groove 22′.

Alternatively, the component according to FIG. 4 may also be an alternative second part 4 of the cachepot 2 according to FIG. 1.

According to a further embodiment of the invention according to FIG. 5, a cachepot 2″ is of one-piece construction, wherein the first capacitive electrode 14′ is arranged on an outer side of the side wall 28 and the second capacitive electrode 15′ is arranged rising from a bottom wall 29. The cachepot 2″ substantially corresponds to the cachepot 2′ according to FIG. 4, but, unlike in FIG. 4, a side projection 30 protrudes from the side wall 28 to fasten the cachepot 2″ to a perpendicular wall.

A further embodiment of the invention according to FIG. 6 provides an intermediate pot 31 with a bottom wall 32 and a side wall 33 extending upright therefrom, which intermediate pot 31 is arranged between a preferably standard commercial one-piece cachepot and the inner pot 3 that accommodates the potting compost. The intermediate pot 31 substantially corresponds to the shape of the second part 5 of the cachepot 2, but, unlike in the embodiment according to FIGS. 1 to 3, a bottom wall 32 is arranged directly on or in the vicinity of the bottom wall 6 of the first part 4 of the cachepot 2. As in the case of the second part 5, the first capacitive electrode 14 and the second capacitive electrode 15 are arranged on the outer side of the intermediate pot 31, and a cable 34 protrudes from each of these electrodes from the opening edge of the intermediate pot 31 to the evaluation device 13 (not shown). Alternatively, the capacitive electrodes 14, 15 can also be arranged on an inner side of the intermediate pot 31.

The intermediate pot 31 differs from the second part 5 substantially in that its shape is adapted to the standard commercial cachepot or to the first part 4 of the cachepot 2. The evaluation device 13 is thus located outside the cachepot 2.

The cachepots 2, 2′, the inner pot 3 and the intermediate pot 31 conventionally have a stop ring 35 on the upper opening edge 23 (FIG. 2).

1. A soil moisture apparatus for plants, with a housing for accommodating the plants (25) and with a moisture measuring device (1) comprising a first capacitive electrode (14, 14′) and a second capacitive electrode (15, 15′) and an evaluation device (13),
wherein the housing is of multi-part construction, comprising
an inner pot (3) for accommodating the potting compost (24) and a cachepot (2, 2′, 2″) that surrounds the inner pot (3) at a bottom wall (26) of the inner pot (3) and/or at a side wall (27) that rises from the bottom wall (26) of the inner pot (3), or
an inner pot (3) for accommodating the potting compost (24) and an intermediate pot (31) that surrounds the inner pot (3) at a bottom wall (26) of the inner pot (3) and/or at a side wall (27) of the inner pot (3), and a cachepot (2, 2′) that surrounds the intermediate pot (31) at a bottom wall (32) and at a side wall (33) of the intermediate pot (31) and
wherein the first capacitive electrode (14, 14′) and the second capacitive electrode (15, 15′) are arranged on an outer side, facing toward the inner pot (3), of the bottom wall and/or the side wall of the cachepot (2, 2′) and/or of the intermediate pot (31).

2. The apparatus according to claim 1, wherein the inner pot (3) consists of a plastics material and the bottom wall and/or the side wall of the inner pot (3) has perforations.

3. The apparatus according to claim 1, wherein the cachepot (2) and/or the intermediate pot (31) are constructed such that the inner pot (3) is insertable with clearance into the cachepot (2) or the intermediate pot (31).

4. The apparatus according to claim 1, wherein the first capacitive electrode (14, 14′) and/or the second capacitive electrode (15, 15′) cover the outer side of the cachepot (2, 2′, 2″) or the intermediate pot (31) over a circumferential angle (φ) of at least 40°.

5. The apparatus according to claim 1, wherein the first capacitive electrode (14, 14′) and/or the second capacitive electrode (15, 15′) take(s) the form of an electrically conductive foil/film or a coating that is bonded to the outer side of the cachepot (2) or intermediate pot (31).

6. The apparatus according to claim 1, wherein the cachepot (2) is of two-part construction with a first part (4) and a second part (5) surrounded by the first part (4), wherein a chamber (12) for accommodating the evaluation device (13) is arranged in a lower portion (11) proximal to the bottom wall of the cachepot (2), and the second part (5) of the cachepot (2) that has the capacitive electrodes (14, 15) is arranged in an upper portion (10) remote from the bottom wall.

7. The apparatus according to claim 1, wherein the second part (5) of the cachepot (2) has opposing grooves (22) on the inner side that each extend from a bottom wall (8) of the second part (5) to an opening edge (23) of the second part (5).

8. The apparatus according to claim 1, wherein the first capacitive electrode (14, 14′) and the second capacitive electrode (15, 15′) are the shape of parts of a cylinder or parts of a cone.

9. The apparatus according to claim 1, wherein electrical cables (34) connected to the respective capacitive electrodes (14, 14′; 15, 15′) protrude from one side of the intermediate pot (31) with a length such that they are connectable to the terminals of the evaluation device (13).

10. The apparatus according to claim 1, wherein the evaluation device (13) is connected to a solar panel (16) and/or to a supercapacitor (17) to power the evaluation device (13).

11. The apparatus according to claim 1, wherein the first capacitive electrode (14, 14′) and/or the second capacitive electrode (15, 15′) cover the outer side of the cachepot (2, 2′, 2″) or the intermediate pot (31) over a circumferential angle (φ) of from 160° to 170°.
